(19) United States
(12) Patent Application Publication
(10) Pub. No.: US 2018/0153477 A1
(43) Pub. Date: Jun. 7, 2018

(54) MULTI-SENSOR STROKE DETECTION

(71) Applicant: Cardiac Pacemakers, Inc., St. Paul, MN (US)

(72) Inventors: Sandra Nagale, Bolton, MA (US); Elizabeth Mary Annoni, White Bear Lake, MN (US); Bryan Allen Clark, Forest Lake, MN (US); Kyle Harish. Srivastava, Saint Paul, MN (US); Pramodsingh Hirasingh Thakur, Woodbury, MN (US); Qi An, Blaine, MN (US); Thomas Christen, Needham, MA (US); Stephen B. Ruble, Lino Lakes, MN (US); Viktoria A. Averina, Shoreview, MN (US); Deepa Mahajan, Roseville, MN (US); Sabrine Ahmed Iqbal, Centreville, VA (US); Edward A. Goldberg, San Diego, CA (US)

(21) Appl. No.: 15/828,144

(22) Filed: Nov. 30, 2017

Related U.S. Application Data
(60) Provisional application No. 62/429,500, filed on Dec. 2, 2016.

(51) Int. Cl. A61B 5/00; A61B 5/0295; A61B 5/021; G06K 9/00; A61B 5/11; G10L 15/02; G16H 50/20

(52) U.S. Cl. CPC A61B 5/7275; A61B 5/0295; A61B 5/0022; A61B 5/021; A61B 5/0004; A61B 5/1118; G10L 15/02; G16H 50/20; G06K 9/00221

(57) ABSTRACT

This document discusses, among other things, systems, devices, and methods for detecting stroke in a patient. A system may comprise a sensor circuit for sensing in a patient at first physiological signal and a second physiological signal or a functional signal. A stroke risk circuit may establish a physiological trend from at least the first physiological signal over time, and generate a stroke risk indicator using the physiological trend and the second physiological or functional signal. Indications of behavioral or cognitive impairment may also be used in stroke risk indicator generation. The system includes an output unit that outputs the stroke risk indicator to a user or a process.

### CLAIM OF PRIORITY

This application claims the benefit of priority under 35 U.S.C. § 119(e) of U.S. Provisional Patent Application Ser. No. 62/429,500, filed on Dec. 2, 2016, which is herein incorporated by reference in its entirety.

### TECHNICAL FIELD

This document relates generally to medical devices, and more particularly, to systems, devices and methods for detecting stroke.

### BACKGROUND

Stroke is one of the leading causes of death and disability in the United States. A stroke occurs when the blood supply to part of the brain is interrupted or severely reduced, thereby depriving brain tissue of oxygen and nutrients. Within minutes, brain cells begin to die. About 85% of strokes are ischemic, as characterized by blockages or narrowing of the arteries, such as caused by blood clots, thereby severely reduced blood flow to the brain.

The faster a person with suspected stroke receives medical attention, the better their prognosis, and the less likely they will be to experience lasting damage or death. In order for a stroke patient to get the best diagnosis and treatment possible, they will need to be treated at a hospital within three hours of their symptoms first appearing. Treatment of stroke may depend on the type of stroke. For ischemic stroke, the treatment may include medications that dissolve blood clots and prevent further ones from forming, such as tissue plasminogen activator (tPA). Device therapy includes self-expandable stent retrievers that may be transvenously placed within the blocked or narrowed blood vessel to trap the clots.

### SUMMARY

Timely detection of an earlier indicator and diagnosis of a stroke is critical to reduce brain damage and death. However, prediction a stroke can be difficult. Usually there tends to be no pain associated with stroke. Patients may therefore miss the prime time for medical attention or the therapeutic window for medication administration. While the diagnosis of stroke may include blood test or imaging tests (e.g., CT scan, MRI scan, carotid ultrasound, or cerebral angiogram), the value of these tests are established provided that the patient can be timely transferred to the hospital. In an ambulatory setting when the patient is away from hospital, the diagnostic imaging may not be available for stroke prediction or for risk stratification.

Patient at risk of stroke may present with confusion, face drooping, arm weakness, trouble with speech, trouble with seeing, trouble with walking such as dizziness and lack of co-ordination, among other signs and symptoms. However, subjective description of these symptoms may be inaccurate and inconsistent. Ambulatory patients may not be able to communicate effectively the symptoms they experience upon a stroke. The information can also be biased due to a need for self-reporting or reliance on caregiver observations. For at least these reasons, the present inventors have recognized, among other things, substantial challenges and a demand for improved system and ambulatory devices to early detection or prevention of stroke.

This document discusses, among other things, systems, devices, and methods for detecting stroke in a patient. A system may comprise a sensor circuit for sensing in a patient at first physiological signal and a second physiological signal or a functional signal. A stroke risk circuit may establish a physiological trend from at least the first physiological signal over time, and generate a stroke risk indicator using the physiological trend and the second physiological or functional signal. Indications of behavioral or cognitive impairment may also be used in stroke risk indicator generation. The system includes an output unit that outputs the stroke risk indicator to a user or a process.

Example 1 is a system for monitoring a patient at risk of a stroke. The system comprises a sensor circuit, a stroke risk circuit, and an output unit. The sensor circuit may be coupled to a first sensor to sense from the patient a first physiological signal and a second sensor to sense from the patient a different second physiological signal or a functional signal. The stroke risk circuit may be communicatively coupled to the sensor circuit, and configured to establish a physiological trend from at least the first physiological signal over time, and generate a stroke risk indicator using the physiological trend and the second physiological or functional signal. The output unit may be configured to output the stroke risk indicator to a user or a process.

In Example 2, the subject matter of Example 1 optionally includes the stroke risk circuit that may be configured to generate the stroke risk indicator using the physiological trend established over a first time window and a second functional signal sensed within a second time window, the first time window starting at a time preceding the second time window.

In Example 3, the subject matter of any one or more of Examples 1-2 optionally includes the stroke risk circuit that may be configured to generate the stroke risk indicator using the physiological trend and the second physiological or functional signal respectively weighted by weight factors indicating respective physiological or functional signal reliability in predicting stroke risk.

In Example 4, the subject matter of any one or more of Examples 1-3 optionally includes the first or second sensor that may be configured to sense the physiological signal including at least one of: a heart rate signal; an atrial rate signal; a heart rate variability signal; a blood pressure signal; a blood pressure variability signal; a body temperature signal; a sympathetic or parasympathetic tone signal; a respiration signal; or a galvanic skin response (GSR) signal.

In Example 5, the subject matter of any one or more of Examples 1-4 optionally includes the first sensor that may include a heart sound (HS) sensor configured to sense a HS signal.

In Example 6, the subject matter of any one or more of Examples 1-4 optionally includes the first sensor that may include a photoplethysmography (PPG) sensor configured to sense a pulse wave propagation parameter.

In Example 7, the subject matter of any one or more of Examples 1-6 optionally includes the second sensor that may be configured to sense a functional signal including at least one of: a posture; a physical activity intensity or duration; a grip strength signal; a gait; or a balance indicator.

In Example 8, the subject matter of any one or more of Examples 1-7 optionally includes the second sensor that may be coupled to a mobile device and configured to detect from the sensed functional signal an indication of behavioral or cognitive impairment. The stroke risk circuit may be configured to generate the stroke risk indicator further using the indication of behavioral or cognitive impairment.

In Example 9, the subject matter of Example 8 optionally includes the mobile device that may be a mobile communication device configured to execute a mobile application for detecting the behavioral or cognitive impairment indication.

In Example 10, the subject matter of Example 8 optionally includes the second sensor that may include a camera configured to capture a facial image of the patient. The mobile device may be configured to detect an indication of facial drooping from the facial image.

In Example 11, the subject matter of Example 8 optionally includes the second sensor that may include a voice recorder configured to record a speech of the patient. The mobile device may be configured to detect an indication of dysarthria from the recorded speech.

In Example 12, the subject matter of Example 8 optionally includes the mobile device that may include a user interface configured to receive text communication from the patient. The mobile device may be configured to detect an indication of dystextia from patient text communication.

In Example 13, the subject matter of any one or more of Examples 1-12 optionally includes the output unit that may be configured to produce an alert based on the stroke risk indicator.

In Example 14, the subject matter of Example 13 optionally includes the output unit that may be configured to produce a recommendation for a diagnostic test or delivery of an anti-stroke therapy based on the stroke risk indicator.

In Example 15, the subject matter of any one or more of Examples 1-14 optionally includes an ambulatory medical device (AMD) communicatively coupled to the first and second sensors. The AMD may include at least a portion of one or more of the sensor circuit, the stroke risk circuit, and the output unit.

Example 16 is a method for monitoring a patient at risk of a stroke using an ambulatory device. The method comprises steps of: sensing, via the ambulatory device, a first physiological signal and a different second physiological signal or a functional signal; establishing a physiological trend of the first physiological signal over time; and generating a stroke risk indicator using the physiological trend and the second physiological or functional signal; and outputting the stroke risk indicator to a user or a process.

In Example 17, the subject matter of Example 16 optionally includes the physiological trend that may be established using the first physiological signal within a first time window. The stroke risk indicator may be generated using a combination of the physiological trend within the first time window and a second functional signal sensed within a second time window, the first time window starting at a time preceding the second time window.

In Example 18, the subject matter of any one or more of Examples 16-17 optionally includes the first or second physiological signal that may include at least one of: a heart rate signal; an atrial rate signal; a heart rate variability signal; a blood pressure signal; a blood pressure variability signal; a heart sound signal; a body temperature signal; a sympathetic or parasympathetic tone signal; a respiration signal; a photoplethysmography signal; or a galvanic skin response (GSR) signal.

In Example 19, the subject matter of any one or more of Examples 16-18 optionally includes the functional signal that may include at least one of: a posture; a physical activity intensity or duration; a grip strength signal; a gait; or a balance indicator.

In Example 20, the subject matter of any one or more of Examples 16-19 optionally include a step of sensing behavioral or cognitive information from the patient via a mobile device, wherein the stroke risk indicator may be generated further using the behavioral or cognitive information.

In Example 21, the subject matter of Example 20 optionally includes the step of sensing the behavioral or cognitive information which may include taking a facial image of the patient, and detecting an indication of facial drooping from the facial image.

In Example 22, the subject matter of any one or more of Examples 20-21 optionally includes the step of sensing the behavioral or cognitive information which may include recording a speech of the patient, and detecting an indication of dysarthria from the recorded speech.

In Example 23, the subject matter of any one or more of Examples 20-22 optionally includes the step of sensing the behavioral or cognitive information which may include receiving text communication from the patient, and detecting an indication of dystextia from patient text communication.

In Example 24, the subject matter of any one or more of Examples 20-23 optionally includes generating an alert based on the stroke risk indicator.

In Example 25, a system may optionally combine any portion or combination of any portion of any one or more of Examples 1-24 to include “means for” performing any portion of any one or more of the functions or methods of Examples 1-24, or a “non-transitory machine-readable medium” including instructions that, when performed by a machine, cause the machine to perform any portion of any one or more of the functions or methods of Examples 1-24.

Detecting a patient risk of stroke using physiological sensors, such as discussed in this document, may improve medical diagnostics of stroke, as well as individualized therapies to improve patient outcome. The systems, devices, and methods discussed in this document may also enhance the performance and functionality of a stroke detection system or device. A device or a system programmed with the sensor-based stroke detection methods can have improved automaticity in medical diagnostics. More efficient device memory or communication bandwidth usage may be achieved by storing or transmitting medical information more relevant to clinical decisions. Additionally, through anti-stroke therapies based on patient individual need and therapy efficacy, battery longevity of an implantable device may be enhanced, or anti-stroke medication volume may be saved.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates generally an example of a stroke monitoring system and portions of an environment in which the system may operate.

FIG. 2 illustrates generally an example of a multi-sensor stroke monitoring system.

FIG. 3 illustrates generally an example of a stroke monitoring system 300 for detecting stroke based at least on behavioral or cognitive impairment.

FIG. 4 illustrates generally an example of a method for detecting stroke in a patient.

FIG. 5 illustrates generally an example of a method for detecting stroke using at least behavioral and cognitive information.

FIG. 6 illustrates generally a block diagram of an example machine upon which any one or more of the techniques (e.g., methodologies) discussed herein may perform.

### DETAILED DESCRIPTION

Disclosed herein are systems, devices, and methods for monitoring a patient to detect stroke. A sensor circuit may sense in a patient physiological and functional signals. A physiological trend may be established from at least the first physiological signal over time. The system can generate a stroke risk indicator using the physiological trend, a functional signal, or additionally an indication of behavioral or cognitive impairment. Clinician may be alerted about the stroke detection, or a therapy be delivered to treat the stroke or to prevent further damages caused by stroke.

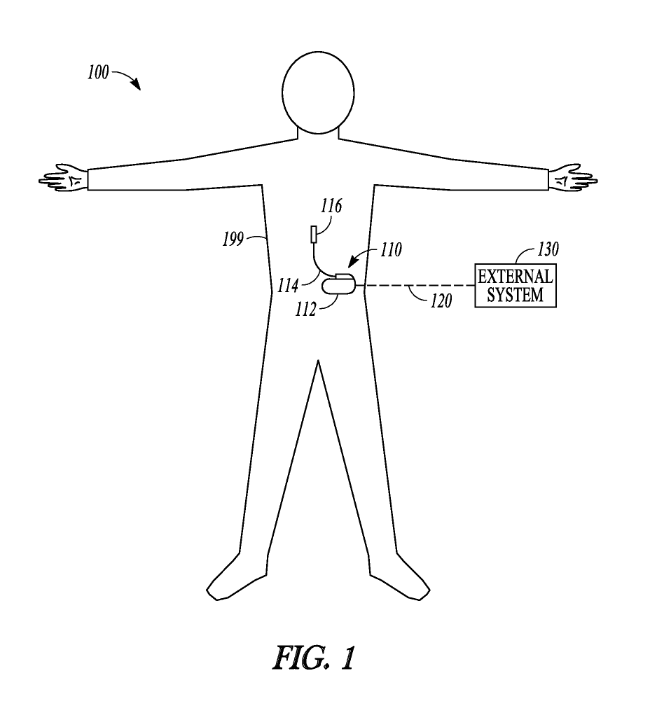

FIG. 1 illustrates generally an example of a stroke monitoring system 100 and portions of an environment in which the system 100 may operate. The stroke monitoring system 100 may include a stroke monitor 110 that may be associated with a body of a patient 199, and an external system 130. A communication link 120 is provided by communication between the stroke monitor 110 and the external system 130.

The stroke monitor 110 may take the form of an ambulatory medical device (AMD) such as an implantable medical device (IMD) 112, a lead system 114, and one or more electrodes 116. The 1 MB 112 may be subcutaneously implanted in a chest, abdomen, or other parts of the body of the patient 199. The IMD 112 may be configured as a monitoring and diagnostic device. The IMD 112 may sense physiological and functional signals in the patient, and predict an impending stroke (e.g., by detecting early indications or signs of stroke) or detect a stroke event. The 1 MB 112 may include a hermetically sealed can that houses a sensing circuitry, control circuitry, communication circuitry, and a battery, among other components. The sensing circuitry of the 1 MB 112 may be configured to sense physiological or functional signals in the patient via sensing electrodes or ambulatory sensors associated with the patient. The physiological or functional signals may contain information about changes in patient cardiovascular, hemodynamic, pulmonary, or neurological responses to physiological or functional changes that are correlated with, or contributing to, development of stroke symptoms. In an example, the IMD 112 may generate a trend of a first physiological signal over a time window, combine the physiological trend with at least a second physiological or functional signal sensed during a different time window, and generate a stroke risk indicator. The IMD 112 may generate an alert of the stroke or pre-stroke indication for the healthcare professionals, or to produce a recommendation for further diagnostic test or treatment.

In addition to patient monitoring and stroke detection, the IMD 112 may additionally include a therapy unit that may generate and deliver one or more therapies to the patient to prevent occurrence of stroke or to treat or control stroke and complications. The therapies may include electrical, magnetic, or other types of therapy. In some examples, the IMD 112 may include a drug delivery system such as a drug infusion pump to deliver drugs to the patient for managing stroke, such as tissue plasminogen activator (tPA) that may dissolve blood clots and restore or improve blood supply to the brain.

Although the discussion herein with respect to the stroke monitoring system 100 focuses on implantable system (e.g., the IMD 112), this is meant only by way of example and not limitation. It is within the contemplation of the inventors and within the scope of this document, that the systems, devices, and methods discussed herein may also be used implemented in, and executed by, a subcutaneous medical devices, wearable medical devices (e.g., watch-like devices, patch-based devices, or other accessories), or other ambulatory medical devices.

The external system 130 may be communicated with the IMD 112 via a communication link 120. The external system 130 may include a dedicated hardware/software system such as a programmer, a remote server-based patient management system, or alternatively a system defined predominantly by software running on a standard personal computer. The external system 130 may control the operation of the IMD 112, such as programming the IMD 112 for detecting stroke and optionally delivering therapies. The external system 130 may additionally receive via the communication link 120 information acquired by IMD 112, such as one or more physiological or functional signals. The external system 130 may include a display for displaying the physiological or functional signals, or alerts, alarms, emergency calls, or other forms of warnings to signal the detection of stroke.

In an example, the external system 130 may include an external data processor configured to analyze the physiological or functional signals received by the 1 MB 112, and to confirm or reject the detection of stroke. Computationally intensive algorithms, such as machine-learning algorithms, may be implemented in and executed by the external data processor, which may process the data retrospectively and provide an individualized prediction of an impending stroke such as to allow the patient to have enough time to react.

The communication link 120 may include one or more communication channels and intermediate devices between the external system and the IMD 112, such as a wired link, a telecommunication link such as an internet connection, or a wireless link such as one or more of an inductive telemetry link, a radio-frequency telemetry link. The communication link 120 may provide for data transmission between the 1 MB 112 and the external system 130. The transmitted data may include, for example, real-time physiological data acquired by the IMD 112, physiological data acquired by and stored in the IMD 112, therapy history data, data indicating device operational status of the IMD 112, one or more programming instructions to the IMD 112 which may include configurations for sensing physiologic signal or stimulation commands and stimulation parameters, or device self-diagnostic test, among others. In some examples, the 1 MB 112 may be coupled to the external system 130 further via an intermediate control device, such as a handheld external remote control device to remotely instruct the IMD 112 to generate electrical stimulation pulses in accordance with selected stimulation parameters produced by the external system 130.

Portions of the 1 MB 112 or the external system 130 may be implemented using hardware, software, firmware, or combinations thereof. Portions of the IMD 112 or the external system 130 may be implemented using an application-specific circuit that may be constructed or configured to perform one or more particular functions, or may be implemented using a general-purpose circuit that may be programmed or otherwise configured to perform one or more particular functions. Such a general-purpose circuit may include a microprocessor or a portion thereof, a microcontroller or a portion thereof, or a programmable logic circuit, or a portion thereof. For example, a “comparator” may include, among other things, an electronic circuit comparator that may be constructed to perform the specific function of a comparison between two signals or the comparator may be implemented as a portion of a general-purpose circuit that may be driven by a code instructing a portion of the general-purpose circuit to perform a comparison between the two signals.

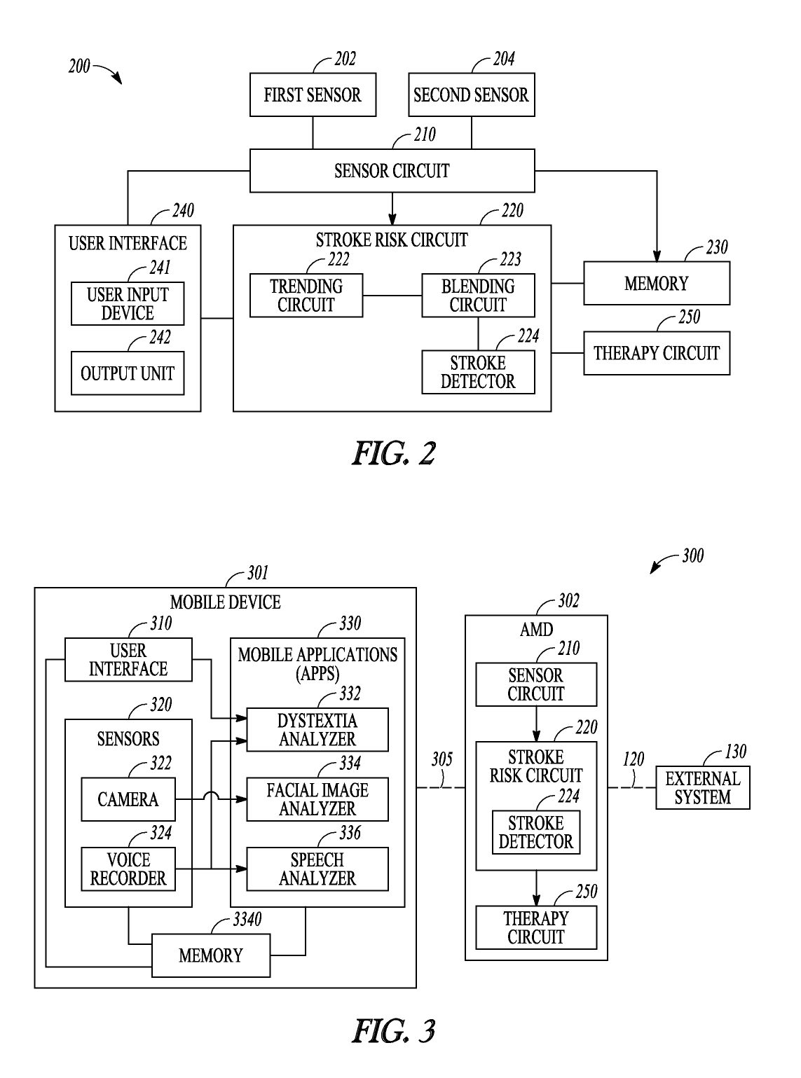

FIG. 2 illustrates generally an example of a multi-sensor stroke monitoring system 200, which can be an embodiment of the stroke monitoring system 100. The multi-sensor stroke monitoring system 200 may include a sensor circuit 210, a stroke risk circuit 220, a memory 230, and a user interface 240. The system 200 may optionally include a therapy circuit 250. In an example, at least a portion of one or more of the sensor circuit 210, the stroke risk circuit 220, the memory 230, the user interface 240, or the optional therapy circuit 250 may be included in an ambulatory device such as the IMD 112, or distributedly implemented between an ambulatory device and an external device such as a programmer or a remote patient management system.

The sensor circuit 210 may include sense amplifiers coupled to two or more sensors, such as a first sensor 202 and a second sensor 204, to sense multiple physiological or functional signals in the patient. The physiological signals may include cardiac, pulmonary, hemodynamic, neural, or biochemical signals. Examples of the physiological signal may include electrocardiograph (ECG), an electrogram (EGM), a heart rate signal, a heart rate variability signal, an intrathoracic impedance signal, an intracardiac impedance signal, an arterial blood pressure signal, a pulmonary artery pressure signal, a RV pressure signal, a LV coronary pressure signal, a blood pressure variability signal, a coronary blood temperature signal, a peripheral body temperature signal, a blood oxygen saturation signal, a heart sound (HS) signal, or a respiration signal (including, for example, respiration rate, tidal volume, minute ventilation, respiratory patterns), a galvanic skin response (GSR) signal, a neural signal such as indicative of sympathetic or parasympathetic tone, among others. Examples of the functional signals may include a posture, a gait, a balance indicator, a locomotion pattern, physical activity intensity or duration, or a grip strength signal, a sleep or awakening state detector, among others. In an example, the first sensor 202 is configured to sense a first physiological signal, and a second sensor 204 is configured to sense a different second physiological signal or a functional signal. In an example, the first sensor may include a photoplethysmography (PPG) sensor configured to sense a pulse wave propagation parameter such as a pulse wave velocity or a pulse wave transit time. The functional signals may include information indicative of a patient behavioral or cognitive impairment, such as textual or verbal communications, speeches, facial expressions, etc. Examples of the behavioral or cognitive assessment and detection of stroke using at least behavioral or cognitive impairment are discussed below, such as with reference to FIG. 3.

In an example, the sensor circuit 210 may be coupled to one or more electrodes such as on the lead system 114 and the can housing of the IMD 112, or one or more implantable, wearable, or other ambulatory sensors to sense the physiological or functional signals. Examples of physiological or functional sensors may include a pressure sensor (e.g., an oscillometry sensor for measuring blood pressure), a flow sensor, a PPG sensor, an impedance sensor, an accelerometer, a microphone sensor, a respiration sensor, a temperature sensor, or a blood chemical sensor, among others. In various examples, an accelerometer may be used to detect an activity intensity or activity duration. A tilt switch, an accelerometer, or a thoracic impedance sensor may be used to detect posture or position. Gyroscope, magnetoresistive sensors, inclinometers, goniometers, electromagnetic tracking system (ETS), sensing fabric, force sensor, strain gauges, and sensors for electromyography (EMG) may be used to measure motion and gaits. In an example, the sensor circuit 210 may be coupled to a device capable of collecting or storing the physiologic information, such as an external programmer, an electronic medical record (EMR) system, or a memory unit, among other data storage devices.

The sense amplifier circuit can pre-process the one or more physiological or functional signals, including, for example, amplification, digitization, filtering, or other signal conditioning operations. The sensor circuit 210 may generate from the preprocessed physiological or functional signals two or more signal metrics representing physiological or functional changes in response to patient disease progression, change in medication, change in health conditions, or change in posture or activity levels. In an example, the sensor circuit 210 may receive a transthoracic impedance signal from the electrodes on the lead system 114 and the can housing of the IMD 112, and generate a signal metric of direct-current (DC) impedance using the transthoracic impedance signal. In another example, the sensor circuit 210 may sense a HS signal from an accelerometer or an acoustic sensor coupled to the IMD 110, and generate two or more HS metrics. Examples of the HS metrics may include intensities of S1, S2, S3, or S4 heart sounds, or timing of the S1, S2, S3, or S4 heart sound with respect to a fiducial point such as a P wave, Q wave, or R wave in an ECG. In an example, the sensor circuit 210 may sense a blood pressure signal via a pressure sensor and generate two or more blood pressure signal metrics which may include systolic blood pressure, diastolic blood pressure, mean arterial pressure, and the timing metrics of these pressure measurements with respect to a fiducial point.

The stroke risk circuit 220 may include circuit sets comprising one or more other circuits or sub-circuits. The circuits or sub-circuits may, alone or in combination, perform the functions, methods, or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

In various examples, the stroke risk circuit 220 may be implemented as a microprocessor circuit, such as a dedicated processor such as a digital signal processor, application specific integrated circuit (ASIC), microprocessor, or other type of processor for processing information including the physiological signals received from the sensor circuit 210. Alternatively, the microprocessor circuit may be a general purpose processor that may receive and execute a set of instructions of performing the functions, methods, or techniques described herein.

As illustrated in FIG. 2, the stroke risk circuit 220, which is communicatively coupled to the sensor circuit 210, may include a trending circuit 222, a blending circuit 223, and a stroke detector 224. Stroke patients may present with one or more trends of elevated heart rate, elevated blood pressure, elevated body temperature, or elevated blood pressure variability, among others. The trending circuit 222 may establish a physiological trend of a signal metric from the first physiological signal over time.

The blending circuit 223 may generate a composite risk score using a combination of the physiological trend and a signal metric from the second physiological signal or a signal metric from the functional signal. In an example, the blending circuit 223 may employ a computation model to perform linear or nonlinear combination of signal metrics. Examples of the computation models may include a linear weighted combination, a nonlinear combination such as a decision tree, a neural network, a fuzzy-logic model, or a multivariate regression model, among others. In an example, the signal metrics may be respectively weighted by weight factors when they are combined. The weight factors indicate respective physiological or functional signal reliability in predicting patient stroke risk. In an example, the reliability may be determined using historical data in the patient, including the physiological or functional signals acquired during stroke episodes in patient medical history. A signal metric with greater and more consistent changes in signal amplitude or signal power is deemed more reliable that another signal metric with smaller changes and larger variance in signal amplitude or signal power. A larger weight may be assigned to the more reliable signal metric than to a less reliable signal metric when establishing a linear or non-linear combination of the signal metrics. In an example, the composite risk score is a numerical risk score computed as weighted sum of individual scores representing likelihood of impending stroke as predicted by individual signal metrics.

Physiological and functional signals may have different time course in responding to stroke. In stroke patients, there may be early cardiac, hemodynamic, or respiratory response prior to an onset of a stroke, when no signs or symptomatic changes in posture, gait, physical activity, or other behavioral or functional changes appear or may be reliably detected by sensors. For example, stroke patients may demonstrate elevated heart rate or blood pressure or elevated body temperature before development of symptoms of functional impairment, such as clumsiness in body movement or gesture, gait disturbances, or difficulty in walking. Taking into account such differences in temporal responses, in an example, the physiological trend of the signal metrics of the physiological signal, such as sensed by the first sensor 202, may be established using a physiological signal sensed during a first time window. Signal metrics from a second functional signal, such as sensed by the second sensor 204, may be generated using a functional signal sensed during a second time window. At least a portion of the first time window may precede second time window. For example, the first time window may start a time preceding the second time window. In an example, the first or second window may have a duration of approximately 1-30 minutes. In another example, the first or second window may have a duration of 1-30 days. The window length may be selected such as to capture both acute changes such as caused by functional signal change (e.g., change in activity or posture) and chronic changes such as caused by disease progression.

The stroke detector 224, coupled to the blending circuit 223, may include a comparator to compare the composite risk score to a predetermined condition, such as a threshold. A stroke risk indicator may be generated when the composite risk score exceeds the threshold. In some examples, the stroke detector 224 may generate the stroke risk indicator using patient demographic information including to age, race and sex, and acquired risk factors include cigarette smoking, hypertension, diabetes, or obesity, among others. In some examples, the stroke detector 224 may generate the stroke risk indicator further using likelihood of an epileptic event further based upon information from patient medical history, such as specific risk factors, conditions, or procedures or treatment that would influence functional or physiological parameters.

The memory 230 may be configured to store sensor signals or signal metrics such as generated by the sensor circuit 210 and the stroke risk indicator. Data storage at the memory 230 may be continuous, periodic, or triggered by a user command or a specified event. In an example, a detection of stroke may trigger the data storage of the physiological signals. In an example, an interrogating device, such as a programmer in the external system 130 as illustrated in FIG. 1 and a remote server-based patient management system, may request access to the stored sensor signals and the stroke risk indicator stored in the memory 230. The requested information may be forwarded to the interrogating device such as via the communication link 120, where the information may be displayed or undergo further analysis, such as to confirm or reject the stroke detection.

The user interface 240 may include an input device 241 and an output unit 242. In an example, at least a portion of the user interface 240 may be implemented in the external system 130. The input device 241 may enable a user to provide parameters for sensing physiological or functional signals, and parameters for detecting stroke risk indicator. The input device 241 may include an input device such as a keyboard, on-screen keyboard, mouse, trackball, touchpad, touch-screen, or other pointing or navigating devices. The output unit 242 may generate a human-perceptible presentation of information including the detection of stroke risk indicator. The output unit 242 may include a display for displaying the information, or a printer for printing hard copies of the information. The information may be presented in a table, a chart, a diagram, or any other types of textual, tabular, or graphical presentation formats, for displaying to a system user. The presentation of the output information may include audio or other media format to inform the system user of the detected physiological events. In an example, the output unit 242 may generate alerts, alarms, emergency calls, or other forms of warnings to signal the system user about patient stroke risk.

The optional therapy circuit 250 may be configured to deliver a therapy to the patient in response to the detection of stroke. In an example, the therapy circuit 250 may control a drug infusion pump to deliver anti-stroke medication, such as tissue plasminogen activator (tPA). In another example, the therapy circuit 250 may deliver a rehabilitative therapy to treat or control side effects of stroke. The rehabilitative therapy may include electrostimulation therapy delivered to a neural target, or tissue or organs with impaired functions. In some examples, the anti-stroke therapy or rehabilitative therapy may be delivered in a closed-loop fashion. The therapy efficacy may be assessed based on sensor feedback. One or more therapy parameters may be adjusted, or drug dosage be tailored, based on the efficacy of the therapy delivered. In some examples, the therapy circuit 250 may provide assistive therapies to maintain adequate cardiorespiratory or hemodynamic support during and after a stroke. Examples of the assistive therapy may include respiratory rate regulation, heart rate regulation, cardiac pacing, or antiarrhythmic therapy, among others.

FIG. 3 illustrates generally an example of a stroke monitoring system 300 for detecting stroke based at least on behavioral or cognitive impairment. The system 300 may include a mobile device 301, an ambulatory medical device (AMD) 302, and an external system 130. Examples of the mobile device 301 may include a smart phone, a wearable device, a portable health monitor, a tablet, a laptop computer, or other types of portable computerized device. The mobile device 301 may be in communication with the AMD 302 via a communication link 305. Examples of the communication link 305 may include a wired connection including universal serial bus (USB) connection, or otherwise cables coupled to communication interfaces on both the mobile device 301 and the AMD 302. Alternatively, the communication link 302 may include a wireless connection including Bluetooth protocol, Bluetooth low energy protocol, Ethernet, IEEE 802.11 wireless, an inductive telemetry link, or a radio-frequency telemetry link, among others.

As illustrated in FIG. 3, the mobile device 301 may comprise a user interface 310 to receive user input, one or more sensors 320 to sense patient functional, behavioral, or biometric information, and a processor executing one or more mobile applications (“apps”) 330 to detect indications of cognitive or behavioral impairment in a patient which are early signs and characteristic symptoms of a stroke. The user interface 310 may include a user input device and a display screen. The input device may include a keyboard, an on-screen keyboard, a touchpad, or a touch-screen, which enables a user to enter texts when prompted to do so. Stroke patients may present with sudden confusion, difficulty in speaking and understanding, or unintelligible verbal and written communication. In an example, one or more questions or instructions may be displayed on the display screen of the user interface 310. The patient user is prompted to answer the questions or perform acts according to the instructions by entering texts using the input device such as the keyboards or the touchpad. The user interface 310 may be coupled to a mobile app of dystextia analyzer 332, which may analyze the patient text communication and generate a dystextia indicator indicating the degree of impairment of patient comprehension and coordination. The text communication may include typing text or selecting from a given list of choices such as prompted on a display. Additionally or alternatively, in a passive mode without prompt, patient spontaneous text communication, such as regular text messages entered via the user interface 310, may be processed by the dystextia analyzer 332 to generate the dystextia indicator. The dystextia indicator may have a numerical or categorical value. For example, a high dystextia score may be generated based on the frequency of unintelligible text messages or the degree of incoherency in the texts entered by the patient. In an example, patient text communication over a period of time may be stored in a memory 340. The dystextia analyzer 332 may trend the dystextia indicators over time using the historical text communications stored in the memory 340, to generate a trend of worsened dystextia. The dystextia indicator, or the dystextia trend, may be forwarded to the stroke detector 224 to detect stroke or predict an impending stroke.

The sensors 320 may include a camera 322 configured to capture a facial image of the patient, or a voice recorder 324 configured to record a speech of the patient. The facial image or the speech may be captured by the respective sensors when the patient is prompted with questions or instructions displayed on the screen of the user interface. For example, the patient may be prompted with instructions to trace some line with his/her finger on the user interface to assess their coordination and comprehension. Other similar guided prompts could be used. Additionally or alternatively, the sensors 320 operate in an unprompted passive mode during normal use of the mobile device 301, such that the camera 322 may capture a facial image when the patient stares at the display screen such as reading a message or browsing web content, or the voice recorder 324 may record a spontaneous speech when the patient answers a phone call.

A mobile app of facial image analyzer 334 may analyze the facial image taken by the camera 322, or a selected portion such as one or more images of eyes, eyebrows, or mouth, to detect an indication of facial drooping. Facial paralysis or drooping is a stroke symptom that may be caused by damaged facial nerve and/or decreased facial muscle tone. Many stroke-associated facial drooping is unilateral, that is, it typically occurs only on one side of the face. Lower eyelid, eyebrow, and corner of the mouth on the affected side of the face are most likely affected area. In an example, the facial image analyzer 334 may compare the facial image to an image template, such as stored in the memory 340, that represents patient normal facial image free of drooping. A dissimilarity measure may be determined such as a spatial distance between image features extracted from the facial image or a portion of the facial image (e.g., eyes or mouth images) and the image features extracted from the image template. Facial drooping may be detected when the dissimilarity measure exceeds a threshold. In another example, the facial image analyzer 334 may detect facial drooping based on asymmetry between the left and right sides of the face, or one or a combination of asymmetry between the left and right eyelids or eyebrows, or between the left and right corners of the mouth. A higher degree of asymmetry may indicate a higher likelihood of facial drooping. The dissimilarity measure or the asymmetry measure of the facial image may be forwarded to the stroke detector 224 to detect stroke or predict an impending stroke.

A mobile app of speech analyzer 336 may process the recorded speech to detect an indication of dysarthria from the recorded speech. Dysarthria, or slurred speech, is a symptom characterized by poor pronunciation of words, mumbling, or a change in speed or rhythm during talking. Stroke may affect different areas of nervous system innervating the muscles for speech production, which may result in impaired movement of the muscles including the lips, tongue, vocal folds, or diaphragm. The speech analyzer 336 may analyze the non-content speech features including, for example, volume, pitch, rhythm, speed, strength, steadiness, range, tone, and accuracy of speech, to generate a dysarthria indicator. The dysarthria indicator may have a numerical or categorical value, indicating a frequency or degree of patterns of continuous breathy voice, irregular breakdown of articulation, mono-pitch, distorted vowels, word flow without pauses, or hypernasality, among others. In an example, patient speech over a period of time may be stored in a memory 340. The speech analyzer 336 may trend the dysarthria indicators over time using the historical speeches stored in the memory 340, and generate a trend of worsened dysarthria. The dysarthria indicator, or the dysarthria trend, may be forwarded to the stroke detector 224 to detect stroke or predict an impending stroke.

In addition to the non-content features, the recorded speech such as produced by the voice recorder 324 may include content-based features that indicate patient cognitive functionality. Similar to the text communication input via the user interface 310, contents of verbal communication from the recorded speech may be analyzed by the dystextia analyzer 332 to generate the dystextia indicator. For example, a high dystextia score may be generated based on the frequency of unintelligible voice messages or conversations in a phone call, or the degree of incoherency in speech. In an example, the dystextia analyzer 332 may generate a trend of worsened dystextia based on both text communications and recorded speech.

The cognitive or behavioral impairment indications, including one or more of the dystextia indicator, the facial drooping or paralysis indicator, or the dysarthria indicator, may be transmitted to the AMD 302 via the communication link 305, and get processed by the stroke risk circuit 220. The stroke risk circuit 220, as previously discussed with reference to the stroke monitoring system 200, may include a stroke detector 224 that generates a stroke risk indicator. The stroke risk indicator may be generated using at least the cognitive or behavioral impairment indications, or optionally further using the physiological and functional signals generated by the sensor circuit 210. It is recognized that at least some stroke patients may manifest cognitive or behavioral impairment that may occur later than a sensor-detectable physiological changes such as cardiac, hemodynamic, or respiratory changes. When taking into account such differences in time course of prediction or temporal responses to stroke, the stroke detector 224 may generate an initial stroke risk indicator based on one or more physiological signal metrics, such as cardiac, hemodynamic, or respiratory signal metrics. The stroke detector 224 may confirm or reject the initial detection using cognitive or behavioral impairment indications acquired subsequent to the physiological signals. A stroke is detected when the initial detection is confirmed by the cognitive or behavioral impairment indications.

The stroke risk indicator, optionally along with the cognitive or behavioral impairment indications, may be forwarded to the external system 130 via the communication link 120 for display or to alert a healthcare provider of the risk of stroke. An optional therapy circuit 250 may deliver drug therapy or electrical therapy in response to the detected risk of stroke, or in response to instructions provided by the healthcare provider via the external system 130.

In some examples, the stroke risk circuit 220 may be implemented within the mobile device 301. The stroke risk circuit 220 may generate the stroke risk indicator using the cognitive or behavioral impairment indications, or optionally further using the physiological and functional signals that may be transmitted to the mobile device 301 from the AMD 302 via the communication link 305. The mobile device 301 may be communicatively coupled to the external system 130 via a communication link such as the communication link 120, and transmit the cognitive or behavioral impairment indications and the stroke risk indication to the external system 130 for display or to alert a healthcare provider of the risk of stroke.

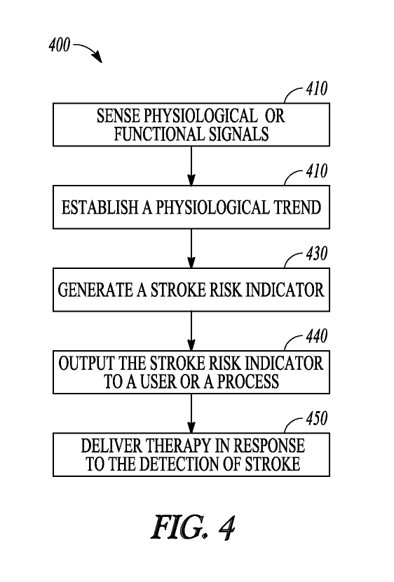

FIG. 4 illustrates generally an example of a method 400 for detecting stroke in a patient. The method 400 may be implemented and executed in an ambulatory medical device such as the IMD 112, or in a remote patient management system such as the external system 130. In an example, the method 500 may be implemented in and executed by the multi-sensor stroke monitoring system 200 as illustrated in FIG. 2.

The method 400 begins at 410 by sensing multiple physiological or functional signals in a patient. The physiological and functional signals may be sensed using respective sensors such as the first and second sensors 202 and 204 as discussed with reference to the stroke monitoring system 200 in FIG. 2. The physiological signals may include cardiac, pulmonary, hemodynamic, neural, or biochemical signals. Examples of the physiological signal may include electrocardiograph (ECG), an electrogram (EGM), a heart rate signal, a heart rate variability signal, an intrathoracic impedance signal, an intracardiac impedance signal, an arterial blood pressure signal, a wave propagation signal indicating pulse wave velocity or a pulse wave transit time, a pulmonary artery pressure signal, a RV pressure signal, a LV coronary pressure signal, a blood pressure variability signal, a coronary blood temperature signal, a peripheral body temperature signal, a blood oxygen saturation signal, a heart sound (HS) signal, or a respiration signal (including, for example, respiration rate, tidal volume, minute ventilation, respiratory patterns), a galvanic skin response (GSR) signal, a neural signal such as indicative of sympathetic or parasympathetic tone, among others. Examples of the functional signals may include a posture, a gait, a balance indicator, a locomotion pattern, physical activity intensity or duration, or a grip strength signal, a sleep or awakening state detector, among others. The functional signals may include information indicative of a patient behavioral or cognitive impairment, such as textual or verbal communications, speeches, facial expressions, etc.

At 420, a physiological signal metric may be trended over time, and a physiological trend can be generated. The signal metric represents physiological or functional changes in response to patient disease progression, change in medication, change in health conditions, or change in posture or activity levels. A stroke risk indicator may be generated at 430 using the physiological trend and the second physiological or functional signal. In an example, a composite risk score may be generated using a combination of the physiological trend and a signal metric from the second physiological signal or a signal metric from the functional signal. The combination may be through a linear or nonlinear computation model. The signal metrics may be respectively weighted by weight factors when they are combined. In an example, the weight factors indicate respective physiological or functional signal reliability in predicting patient stroke risk. In an example, the composite risk score may be a numerical risk score computed as weighted sum of individual scores representing likelihood of impending stroke as predicted by individual signal metrics.

At 440, the detection of the stroke may be output to a user or a process. In an example, a human-perceptible presentation of information, including the stroke risk indicator, may be generated and displayed such as on the output unit 242 of a user interface 240 as illustrated in FIG. 2. In an example, alerts, alarms, emergency calls, or other forms of warnings may be generated to signal an earlier detection of stroke.

The method 400 may optionally include a step 450 for delivering a therapy to the patient in response to the detection of stroke. The therapy may include drug therapy such as delivery of anti-stroke medications through a drug infusion pump device, and/or rehabilitative therapy to control side effects of stroke, such as electrostimulation therapy delivered to a neural target, or tissue or organs with impaired functions. The anti-stroke therapy or rehabilitative therapy may be delivered in a closed-loop fashion. In some examples, assistive therapies may be delivered at 450 to maintain adequate cardiorespiratory or hemodynamic support during and after a stroke.

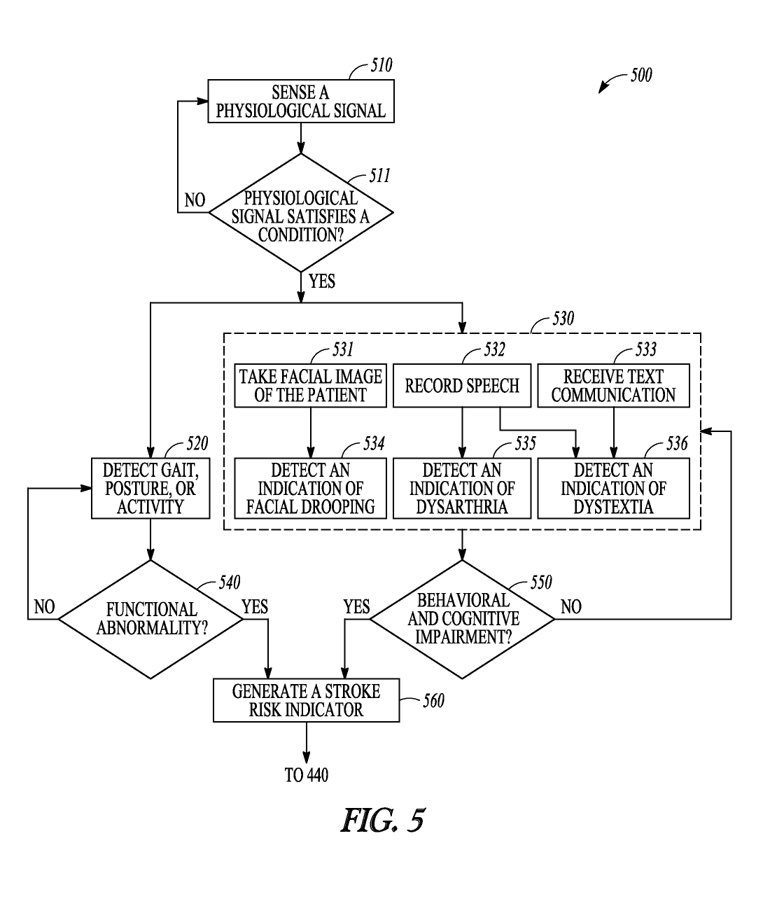

FIG. 5 illustrates generally an example of a method 500 for detecting stroke using at least behavioral and cognitive information. The method 500 may be an embodiment of the method 400, and may be implemented in and executed by the stroke monitoring system 300 as in FIG. 3.

The method 500 begins at 510 by sensing one or more physiological signals such as via an ambulatory physiological sensor 202. The physiological signal may be processed and a physiological trend may be generated as discussed previously with respect to step 420 of the method 400. At 511, the physiological signal may be evaluated against a specified condition to decide whether a sign of stroke is presented. One or more signal metrics generated from the physiological signals may be compared to respective thresholds. In some stroke patients, functional symptoms and cognitive or behavioral impairment may occur later in time than physiological changes such as cardiac, hemodynamic, or respiratory changes. If the pre-determined condition is satisfied (e.g., one or more signal trends of heart rate, blood pressure, blood pressure variability, or body temperature demonstrate an increase that exceeds respective thresholds), the initial detection of physiological abnormality may trigger the functional assessment at 520 and behavioral and cognitive assessment at 530.

At 520, functional signals such as gait, posture, or physical activity may be detected such as by using the second sensor 204 as illustrated in FIG. 2. In an example, the functional signal may be sensed at a time behind the physiological signal to taking into account the difference between physiological response to and functional manifestation of stroke. For example, the physiological signal may be sensed during a first time window preceding in time than a second time window during which the functional signals are sensed. In an example, the first time window may have an earlier onset time than the second time window. Functional abnormality may be detected from the functional signals at 540, such as clumsiness in body movement or gesture, gait disturbances, or difficulty in walking.

At 530, behavioral and cognitive assessment may be performed such as using sensors or input devices included in a mobile device 301 as illustrated in FIG. 3. By way of example and not limitation, the behavioral and cognitive assessment may include taking a facial image at 531 such as via a camera 322, recording patient speech at 532 such as via the voice recorder 324, or acquiring patient text communication at 533 such as via the user interface 310. The facial image, speech, or the text communication may be obtained when patient is prompted through the user interface or in an unprompted passive mode during daily activities. The facial image may be processed at 534 to detect an indication of facial drooping, such as based on a dissimilarity measure between the facial image and an image template, or based on an asymmetry between the left and right eyelids or eyebrows or between the left and right corners of the mouth.

The recorded speech may include both non-content features and content-based features. The non-content features indicate muscles functions for speech production (e.g., volume, pitch, rhythm, speed, strength, steadiness, range, tone, and accuracy of speech). The content-based features indicate patient cognitive functionality. At 535, the non-content features of the recorded speech may be used to generate a dysarthria indicator indicating frequency or degree of patterns of slurred speech. The content-based features of the recorded speech may be used to derive a dystextia indicator at 536, such as based on the frequency of unintelligible voice messages or conversations in a phone call, or the degree of incoherency in speech. The dystextia indicator indicates a degree of impairment of patient comprehension and coordination. In addition to the speech content, the dystextia indicator at 536 may also be generated using text communication such as text messages entered by the patient. The text communication may be analyzed at 536 to determine a frequency of unintelligible text messages or the degree of incoherency in the texts.

At 550, behavioral and cognitive impairment may be determined based on one or more of the dystextia indicator, the facial drooping or paralysis indicator, or the dysarthria indicator. In an example, these indicators may be represented respectively by numerical scores, such as a dystextia score, a facial drooping score, or a dysarthria score. A composite behavioral and cognitive score may be generated using a combination of these scores, and impairment is detected at 550 if the composite score exceeds a specified threshold.

At 560, a stroke risk indicator may be generated if the functional abnormality is detected at 540, or the behavioral cognitive impairment is detected at 550. A stroke is detected when the initial detection based on the physiological symptoms at 511 is confirmed by the cognitive or behavioral impairment and the functional abnormality. The stroke risk indicator, optionally along with the physiological, the functional, and the cognitive or behavioral signals, may be output to a user or a process at 440. The stroke risk indicator may optionally trigger delivery of therapy.

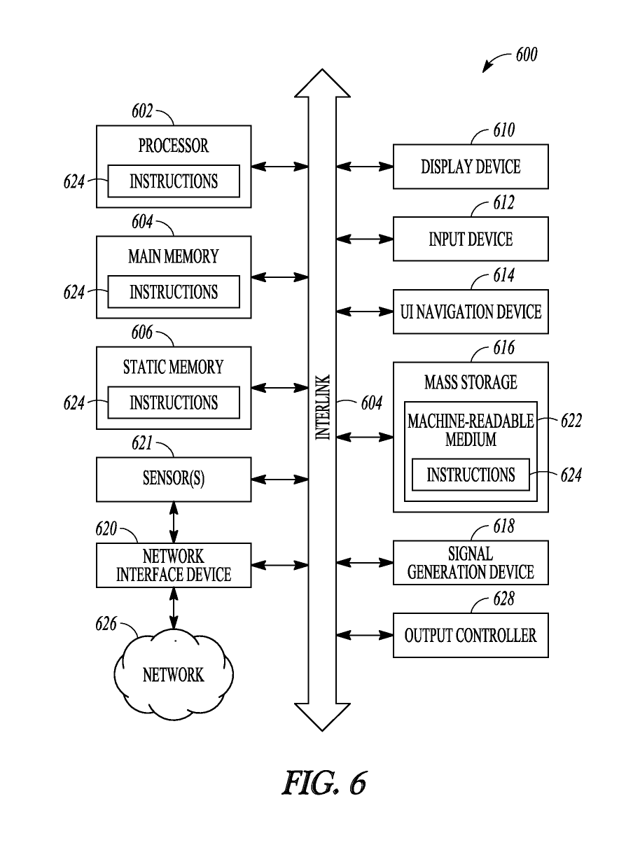

FIG. 6 illustrates generally a block diagram of an example machine 600 upon which any one or more of the techniques (e.g., methodologies) discussed herein may perform. Portions of this description may apply to the computing framework of various portions of the LCP device, the IMD, or the external programmer.

In alternative embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 600 may include a hardware processor 602 (e.g., a central processing unit (CPU), a graphics processing unit (GPU), a hardware processor core, or any combination thereof), a main memory 604 and a static memory 606, some or all of which may communicate with each other via an interlink (e.g., bus) 608. The machine 600 may further include a display unit 610 (e.g., a raster display, vector display, holographic display, etc.), an alphanumeric input device 612 (e.g., a keyboard), and a user interface (UI) navigation device 614 (e.g., a mouse). In an example, the display unit 610, input device 612 and UI navigation device 614 may be a touch screen display. The machine 600 may additionally include a storage device (e.g., drive unit) 616, a signal generation device 618 (e.g., a speaker), a network interface device 620, and one or more sensors 621, such as a global positioning system (GPS) sensor, compass, accelerometer, or other sensor. The machine 600 may include an output controller 628, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 616 may include a machine readable medium 622 on which is stored one or more sets of data structures or instructions 624 (e.g., software) embodying or utilized by any one or more of the techniques or functions described herein. The instructions 624 may also reside, completely or at least partially, within the main memory 604, within static memory 606, or within the hardware processor 602 during execution thereof by the machine 600. In an example, one or any combination of the hardware processor 602, the main memory 604, the static memory 606, or the storage device 616 may constitute machine readable media.

While the machine readable medium 622 is illustrated as a single medium, the term “machine readable medium” may include a single medium or multiple media (e.g., a centralized or distributed database, and/or associated caches and servers) configured to store the one or more instructions 624.

The term “machine readable medium” may include any medium that is capable of storing, encoding, or carrying instructions for execution by the machine 600 and that cause the machine 600 to perform any one or more of the techniques of the present disclosure, or that is capable of storing, encoding or carrying data structures used by or associated with such instructions. Non-limiting machine readable medium examples may include solid-state memories, and optical and magnetic media. In an example, a massed machine readable medium comprises a machine readable medium with a plurality of particles having invariant (e.g., rest) mass. Accordingly, massed machine-readable media are not transitory propagating signals. Specific examples of massed machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 624 may further be transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a low power side area network (LPWAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802.16 family of standards known as WiMax®), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 600, and includes digital or analog communications signals or other intangible medium to facilitate communication of such software.

Various embodiments are illustrated in the figures above. One or more features from one or more of these embodiments may be combined to form other embodiments.

The method examples described herein can be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A system for monitoring a patient at risk of a stroke, the system comprising:
a sensor circuit, coupled to a first sensor to sense from the patient a first physiological signal and a second sensor to sense from the patient a different second physiological signal or a functional signal;
a stroke risk circuit communicatively coupled to the sensor circuit, the stroke risk circuit configured to:
establish a physiological trend from at least the first physiological signal over time; and
generate a stroke risk indicator using the physiological trend and the second physiological or functional signal; and
an output unit configured to output the stroke risk indicator to a user or a process.

2. The system of claim 1, wherein the stroke risk circuit is configured to generate the stroke risk indicator using the physiological trend established over a first time window and a second functional signal sensed within a second time window, the first time window starting at a time preceding the second time window.

3. The system of claim 1, wherein the stroke risk circuit is configured to generate the stroke risk indicator using the physiological trend and the second physiological or functional signal respectively weighted by weight factors indicating respective physiological or functional signal reliability in predicting stroke risk.

4. The system of claim 1, wherein the first or second sensor is configured to sense the physiological signal including at least one of:
a heart rate signal;
an atrial rate signal;
a heart rate variability signal;
a blood pressure signal;
a blood pressure variability signal;
a heart sound signal;
a body temperature signal;
a sympathetic or parasympathetic tone signal;
a respiration signal;
a photoplethysmography signal; or
a galvanic skin response (GSR) signal.

5. The system of claim 1, wherein the second sensor is configured to sense a functional signal including at least one of:
a posture;
a physical activity intensity or duration;
a grip strength signal;
a gait; or
a balance indicator.

6. The system of claim 1, wherein:
the second sensor is coupled to a mobile device configured to detect from the sensed functional signal an indication of behavioral or cognitive impairment; and
the stroke risk circuit is configured to generate the stroke risk indicator further using the indication of behavioral or cognitive impairment.

7. The system of claim 6, wherein the second sensor includes a camera configured to capture a facial image of the patient, and the mobile device is configured to detect an indication of facial drooping from the facial image.

8. The system of claim 6, wherein the second sensor includes a voice recorder configured to record a speech of the patient, and the mobile device is configured to detect an indication of dysarthria from the recorded speech.

9. The system of claim 6, wherein the mobile device includes a user interface configured to receive text communication from the patient, and the mobile device is configured to detect an indication of dystextia from patient text communication.

10. The system of claim 1, wherein the output unit is configured to produce an alert based on the stroke risk indicator.

11. The system of claim 1, comprising an ambulatory medical device (AMD) communicatively coupled to the first and second sensors, the AMD including at least a portion of one or more of the sensor circuit, the stroke risk circuit, and the output unit.

12. A method for monitoring a patient at risk of a stroke using an ambulatory device, the method comprising:
sensing, via the ambulatory device, a first physiological signal and a different second physiological signal or a functional signal;
establishing a physiological trend of the first physiological signal over time; and
generating a stroke risk indicator using the physiological trend and the second physiological or functional signal; and
outputting the stroke risk indicator to a user or a process.

13. The method of claim 12, wherein:
the physiological trend is established using the first physiological signal within a first time window; and
the stroke risk indicator is generated using a combination of the physiological trend within the first time window and a second functional signal sensed within a second time window, the first time window starting at a time preceding the second time window.

14. The method of claim 12, wherein the first or second physiological signal includes at least one of:
a heart rate signal;
an atrial rate signal;
a heart rate variability signal;
a blood pressure signal;
a blood pressure variability signal;
a heart sound signal;
a body temperature signal;
a sympathetic or parasympathetic tone signal;
a respiration signal;
a photoplethysmography signal; or
a galvanic skin response (GSR) signal.

15. The method of claim 12, wherein the functional signal includes at least one of:
a posture;
a physical activity intensity or duration;
a grip strength signal;
a gait; or
a balance indicator.

16. The method of claim 12, further comprising sensing behavioral or cognitive information from the patient via a mobile device, wherein the stroke risk indicator is generated further using the behavioral or cognitive information.

17. The method of claim 16, wherein sensing the behavioral or cognitive information includes taking a facial image of the patient, and detecting an indication of facial drooping from the facial image.

18. The method of claim 16, wherein sensing the behavioral or cognitive information includes recording a speech of the patient, and detecting an indication of dysarthria from the recorded speech.

19. The method of claim 16, wherein sensing the behavioral or cognitive information includes receiving text communication from the patient, and detecting an indication of dystextia from patient text communication.

20. The method of claim 16, wherein comprising generating an alert based on the stroke risk indicator.
